(19) United States
(12) Patent Application Publication
(10) Pub. No.: US 2007/0031862 A1
(43) Pub. Date: Feb. 8, 2007

(54) PREPARATION OF PLASTIC SUPPORTS FOR BIOCHIPS

(75) Inventors: Boris K. Chernov, Burr Ridge, IL (US); Julia B. Golova, Burr Ridge, IL (US); Darrell P. Chandler, Yorkville, IL (US)

Correspondence Address: BARNES & THORNBURG LLP, P.O. BOX 2786, CHICAGO, IL, 60690-2786, US

(73) Assignee: The University of Chicago, Chicago, IL (US)

(21) Appl. No.: 11/425,667

(22) Filed: Jun. 21, 2006

Related U.S. Application Data
(60) Provisional application No. 60/706,494, filed on Aug. 8, 2005.

(51) Int. Cl. C12Q 1/68; C12M 1/34

(52) U.S. Cl. 435/6; 435/287.2; 427/2.11; 977/924

(57) ABSTRACT

Plastic substrates, including poly(methyl methacrylate) (PMMA) and poly(ethylene terephthalate) (PET) slides, may be modified with methods described herein and subsequently used in the manufacture or formation of biochips, or other microarrays. Illustrative methods include modifying the surface of the plastic substrates by covalent attachment of unsaturated acid derivatives that include a primary amine group for reacting with functional groups on the surface of the plastic substrate. Further illustrative methods include modifying the surface of the plastic substrates by a cleaning procedure that results in improved adhesive properties for subsequent printing. After being modified, the substrates described herein may be used to prepare 2D and 3D microarrays by printing using conventional methods.

This patent application claims priority from U.S. Provisional Patent Application No. 60/706,494, filed Aug. 8, 2005.

This invention was made in part under Contract No. W-31-109-ENG-38 between the U.S. Department of Energy and the University of Chicago representing Argonne National Laboratory. The U.S. Government may have certain rights in this invention.

### BACKGROUND

Biochips are becoming increasing important to state-of-the-art diagnostic methods. Such biochips may include a wide variety of biologically important molecules, including single stranded and double stranded nucleic acids, nucleic acid hybridization probes, proteins, peptides, carbohydrates, lipids, and others. Conventional biochips are generally manufactured on glass supports i.e. glass slides. However, in certain applications, glass supports do not possess the necessary or desirable mechanical properties, including mechanical strength, impact resistance, toughness, and the like. Accordingly, alternative supports are needed for the preparation of biochips and related technologies.

In addition, although biochips have been in the marketplace in various formats for several years, the emergence of new technologies, including microfluidic technology and nanotechnology provides additional applications for biochips. In particular, improvements in microfluidic technology may have a revolutionary impact on the next generation of laboratory on a chip (“lab-on-chip”) assays based on biochip technology, particularly as nanotechnology moves into wider applications. Novel biochips and supports therefor are yet needed to meet the requirements of these and other emerging technologies.

### SUMMARY

Methods for preparing a plastic support that may be subsequently printed with one or more biomolecules or a micorarray of gel drops loaded with biomolecules to form biochips are described. The plastic support is used to prepare a microarray of biomolecules or a microarray of gel drops loaded with biomolecules, such as for laboratory on a chip assays. One illustrative method includes the steps of:
(a) reacting the plastic support with an unsaturated carboxylic acid derivative having a primary amine functional group; and
(b) printing one or more biomolecules or microarrays of gel forming mixtures loaded with one or more biomolecules on the plastic support; and
(c) covalently attaching the one or more biomolecules or gel forming mixtures copolymerized with biomolecules to the plastic support.

Another illustrative method includes the steps of:
(a) washing the plastic support with an organic solvent; and
(b) printing a microarray of gel forming mixtures loaded with one or more biomolecules on the plastic support; and
(c) attaching a gel drops microarray to the plastic support during polymerization of the gel forming mixtures.

The methods described herein may be used to prepare two-dimensional (2D) or three-dimensional (3D) biochips. In either case, conventional printing methods may be used to prepare the biochip. It is appreciated that in the case of methods for preparing 2D biochips, the plastic support includes a functional group on the surface or accessible from the surface that is capable of reacting with a functional group included on the biomolecule to make a covalent bond.

In the case of 3D biochips, one illustrative aspect of the methods described herein includes printing the one or more biomolecules using a conventional gel-drop method of printing wherein the one or more biomolecules is admixed with a gel-forming mixture. It is appreciated that in the case of methods for preparing 3D biochips, the plastic support may include a functional group on the surface or accessible from the surface that is capable of reacting with a functional group included in the gel forming mixture to make a covalent bond. Alternatively, the plastic support has been cleaned or washed as described herein to prepare a more adhesive surface for attaching gel drops bearing the biomolecules.

In another illustrative embodiment, biochips prepared on plastic supports using the methods presented herein are described. In one aspect, the biochips are 2D biochips. Biomolecules are covalently attached to the plastic support using an olefin polymerization reaction. In another aspect, the biochips are 3D biochips. Biomolecules are covalently attached to a gel drop that is either covalently or adhesively attached to a plastic support. Biomolecules are covalently attached to the gel drop using an olefin polymerization reaction.

The following definitions are used herein:

Biochip, array, microarray: a predetermined arrangement of molecules relative to each other, connected to a support; also referred to as a microchip, DNA chip, DNA microarray, DNA array, peptide chip, or peptide array. Illustratively, the array is a predetermined arrangement of biological molecules such as DNA fragments, peptides, proteins, lipids, drugs, affinity ligands, and the like.

Bioprobe: synthetic oligonucleotide, DNA fragment, protein and the like.

EDTA: ethylendiaminetetraacetic acid.

FITC: fluorescein-5-isothiocyanate

Hybridization: the process of joining two complementary strands of DNA or one each of DNA and RNA to form a double-stranded molecule.

Microfluidic devices: a set of devices produced by technologies that control the flow of micro, nano, or even picoliter amounts of liquids or gases in a miniaturized system.

Microarray printing: dispensing a known volume at each selected array position by tapping a capillary dispenser or solid pin on a support in order to deposit a defined volume of solution.

Oligonucleotide: A nucleotide sequence (DNA or RNA) having about 6 or more nucleotides, and illustratively in the range from about 6 to about 100 nucleotides.

PCR: Polymerase chain reaction. A method used to make multiple copies of DNA.

Plastic: synthetic or semisynthetic organic based polymeric materials that may illustratively be molded, or extruded into an object, bead, film or filaments, or used for making coatings or adhesives, including but not limited to poly(methyl methacrylate) (PMMA), poly(ethylene terephthalate) (PET), polystyrene, combinations thereof, copolymers thereof with other block polymers, blended polymers thereof with other polymers, and the like.

SSPE: saline-sodium phosphate-EDTA buffer.

Support: Insoluble, functionalized, polymeric material (glass, plastic, silicon etc.), to which elements may be attached.

Tween: C64H124O26, non-ionic detergent.

UV: ultraviolet.

### BRIEF DESCRIPTION OF THE DRAWINGS

FIG. 1 shows chemical modifications of poly(methyl methacrylate) (PMMA) or poly(ethylene terephthalate) (PET) supports. PMMA (1) and/or PET (4) supports are treated with an aqueous solution of monomethacrylamide derivatives of aminoalkanes, such as 1,6-diaminohexane derivative (2). Modified supports (3) and/or (5) containing methacrylic functional groups are formed. Treatment of plastic supports with fluorescent labeled diaminoalkanes, such as 1,6-diaminohexane derivative (6) are used for qualitative and quantitative determination of the modification procedure efficiency.

FIG. 2 shows a scheme for manufacturing 3D microarrays on plastic supports, such as by gel-drop methods, including the steps of (a) washing or cleaning the support thereby improving the adhesive properties of the plastic surface, (b) chemical modification of the support, where M illustratively represents the functional group of the modification used to covalently attach the biomolecules or gel drops, including an unsaturated carboxylic acid group, such as a methacrylate or acrylate group, and (c) printing a copolymerization mixture containing methacrylated oligonucleotides as drops on the plastic support and polymerizing the gel drops, such as by UV exposure.

FIG. 3 shows kinetics of nonequilibrium hybridization of fluorescent labeled 20-mer target oligonucleotide on gel drops array with immobilized 20-mer oligonucleotide probes manufactured on PET slide (diamonds) and glass slide (quadrants).

### DETAILED DESCRIPTION

Methods for preparing plastic supports that may be subsequently printed with one or more biomolecules are described. In one embodiment, the plastic support is used to prepare a microarray of biomolecules for laboratory on a chip assays. An illustrative method includes the steps of:
(a) reacting the plastic support with an unsaturated carboxylic acid derivative having a primary amine functional group;
(b) printing one or more biomolecules or microarray of gel forming mixtures loaded with one or more biomolecules on the plastic support; and
(c) covalently attaching biomolecules or gel drops copolymerized with biomolecules to the plastic support.

Another illustrative method includes the steps of:
(a) washing a plastic support with an organic solvent;
(b) printing a microarray of gel forming mixtures loaded with one or more biomolecules on the plastic support; and
(c) attaching a gel drops microarray to the plastic support during polymerization of gel forming mixtures.

Biochips on plastic slides can be produced in two formats: two dimensional (2D) or three dimensional (3D). In the first case, a biochip is manufactured by spotting functional groups on the surface of plastic slides prepared as described herein. Following spotting, UV exposure provides formation of covalent bonds between biomolecules (probes) and the plastic surface through radical polymerization reactions. 3D biochip formation is performed by copolymerization when gel forming solutions mixed with methacrylated bioprobes (DNA fragments, proteins and the like) are placed as spots on the surface of a plastic slide, and then after UV exposure, polymerization of gel drops and their attachment to the slide take place.

The methods described herein may be used to prepare two-dimensional (2D) or three-dimensional (3D) biochips. In either case, conventional printing methods may be used to prepare the biochip.

In the case of 3D biochips, one illustrative aspect of the methods described herein includes printing the one or more biomolecules using a conventional gel-drop method of biochip manufacturing.

In another illustrative embodiment, biochips prepared on plastic supports using the methods set forth herein are described. In one aspect, the biochips are 2D biochips. The biomolecules are covalently attached to the plastic support using an olefin polymerization reaction. In another aspect, the biochips are 3D biochips. The biomolecules are covalently attached to a gel drop that is either covalently or adhesively attached to the plastic support. In either aspect, the biomolecules are covalently attached to the gel drop using an olefin polymerization reaction.

In an illustrative aspect, the plastic support comprises poly(methyl methacrylate) (PMMA), poly(ethylene terphthalate) (PET), copolymers, block and blended copolymers of the foregoing, including copolymers of polysterene, polyurethene, poly (ethylene oxide). It is appreciated that any plastic support that is reactive with a primary amine may be used in the methods described herein for preparing 2D miscroarrays. It is further appreciated that any plastic that exhibits the properties of improved adhesion after washing or cleaning with organic solvents may be used in the methods described herein for preparing 3D micorarrays.

It is to be understood that the plastic supports described herein include a variety of physical forms, including slides, beads, films, and the like. Illustratively, commercially available plastic slides are used in the methods described herein to prepare microarrays of biomolecules.

Although a large number of different plastics are available, it is understood that the ultimate choice of material may depend on the final application. Illustratively for biochip manufacturing, selection criteria include transparency, chemical resistance, biocompatibility and preselected surface properties, including hydrophilic or hydrophobic properties. Illustratively, thermoplastics comprising polystyrene, poly(methyl methacrylate), poly(ethylene terephthalate) (PET), poly(carbonate), cycloolefin copolymers with polypropylene or polyethylene polymers, block co-polymers of varying chemical composition, and blend polymers of these and other polymers are suitable.

The one or more biomolecules to be included on the biochips described herein may be printed using conventional techniques.

Gel drops microarray manufacturing based on pin-printing techniques are described in Arenkov et al. (2000): Mirzabekov et al (2001). Pin-printed technology is used by several large array producing companies such as Aglient Technologies, TeleChem International Inc., Apply Biochemistry Inc., Perkin-Elmer.

In one aspect, the biomolecules are printed onto a plastic support prepared by the methods described herein, then subsequently covalently attached to the plastic support. In this illustrative aspect, 2D microarrays are prepared by chemically modifying the plastic support to include functional groups that can react with and covalently attach to the biomolecules. Illustratively, the functional groups are unsaturated acids and derivatives thereof that may form covalent bonds or polymers with the biomolecules. Illustrative unsaturated acids and derivatives thereof include acrylic, methacrylic, crotonic, butenoic, fumaric, maleic, and other acids and amide derivatives thereof. The derivatives also illustratively include a linker separating the primary amine functional group from the unsaturated carboxylic acid derivative. Illustrative linkers include bivalent linear or branched alkylene, cycloalkylene, alkenylene, alkynylene, or phenylene groups, or a combination thereof in forming a linker. In another aspect, the linker is an alkylene linker. Such alkylene linkers may include any number of carbon atoms, and illustratively includes from 2 to about 10 in a linear fragment. It is appreciated that such alkylene linkers may be branched, or portions of such alkylene linkers may be cyclized. In one variation, the alkylene linker is a straight chain of the formula —(CH₂)ₙ—, where n is an integer from 2 to about 10, or from 4 to about 8. In another aspect, the unsaturated carboxylic acid derivative is an amide of acrylic or methacrylic acid.

In another aspect, the modifying compounds include a primary amine functional group. It is appreciated that such a functional group is capable of reacting with other functional groups present on the surface or accessible from the surface of the plastic support to be prepared according to the methods described herein. For example, primary amines may react with carboxylate bonds present on the surface of PET, polycarbonate, and like polymers, ester bonds present on the surface of polyacrylate, polymethacrylate, and like polymers, urea bonds present on urethanes, and like polymers, and others present on the surface of the plastic support. In another aspect, the primary amine functional group is covalently attached to the unsaturated carboxylic acid derivative through a bivalent linear or branched alkylene, cycloalkylene, alkenylene, alkynylene, or phenylene group, or a combination thereof. In another aspect, the primary amine functional group is covalently attached to the unsaturated carboxylic acid derivative through a bivalent alkylene, group.

In another aspect, the biomolecules are mixed with a gel forming mixture that includes a gel forming polymer and an optional cross-linking agent. Illustrative gel forming mixtures are described in U.S. Pat. No. 6,927,025, and references cited therein.

After printing, the gel forming mixture including the biomolecules is polymerized spontaneously or in a subsequent step to form a gel drop. In this illustrative aspect, 3D microarrays are prepared. Illustratively, the gel forming polymer includes unsaturated acids and derivatives thereof that may form polymers with the biomolecules. Illustrative unsaturated acids and derivatives thereof include acrylic, methacrylic, crotonic, butenoic, funaric, maleic, and other acids and amide derivatives thereof. The derivatives also illustratively include a linker separating the primary amine functional group from the unsaturated carboxylic acid derivative. Illustrative linkers include bivalent linear or branched alkylene, cycloalkylene, alkenylene, alkynylene, or phenylene groups, or a combination thereof in forming a linker. In another aspect, the linker is an alkylene linker. Such alkylene linkers may include any number of carbon atoms, and illustratively includes from 2 to about 10 in a linear fragment. It is appreciated that such alkylene linkers may be branched, or portions of such alkylene linkers may be cyclized. In one variation, the alkylene linker is a straight chain of the formula —(CH₂)ₙ—, where n is an integer from 2 to about 10, or from about 4 to about 8.

In another embodiment of gel drop microarrays, (also known as copolymerization microarrays) bioprobes, including oligonucleotides, proteins, and the like, are mixed with an unpolymerized gel forming mixture, applied as one or more spots on the plastic support, and then the gel forming mixture is polymerized to produce a gel drop microarray attached to the plastic surface, using otherwise conventional procedures. Fixing of gel elements on the support may be provided by first modifying the support with unsaturated carboxylic acid, such as methacrylic acid, functional groups able to participate in the copolymerization process. Fixing of gel elements on the support may also be provided by first increasing the adhesive properties of the plastic supports by washing or cleaning the surface with a selected organic solvent as described herein.

In the various embodiments described herein, the biomolecules should also be modified to facilitate covalent attachment with the gel drop or the chemically modified plastic support surface. In one aspect, the biomolecules chemically modified with unsaturated acids and derivatives thereof including acrylic, methacrylic, crotonic, butenoic, fumaric, maleic, and other acids, and amide derivatives thereof.

In another embodiment, covalent attachment of the one or more biomolecules to either the chemically modified plastic support surface, or to the gel drop is performed by UV radiation. It is appreciated that methacrylic acid and derivatives thereof are suitable for such UV polymerization initiation. In another embodiment, covalent attachment of the one or more biomolecules to either the chemically modified plastic support surface, or to the gel drop is performed by radical polymerization, including heat and chemically initiated processes.

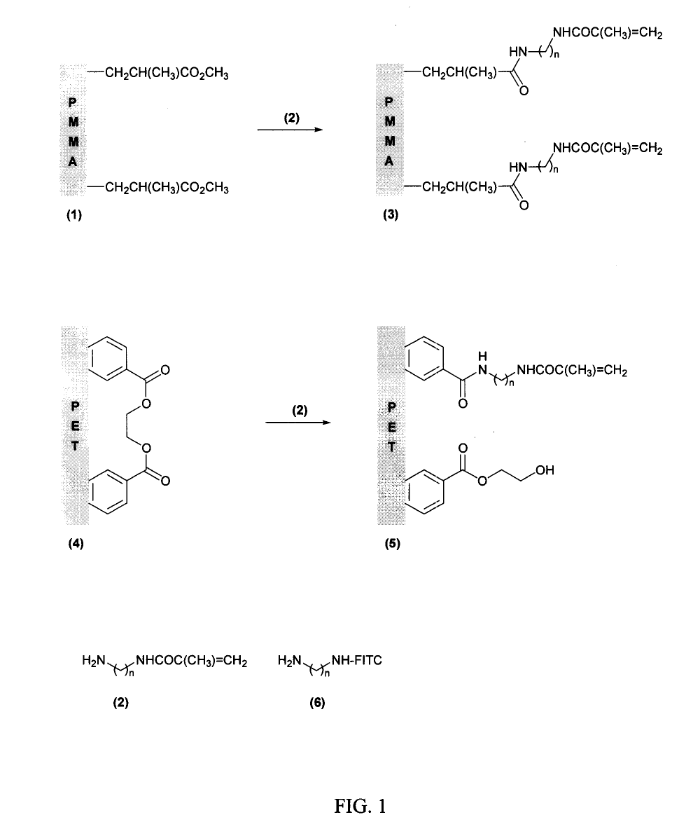

Exemplary embodiments are shown in FIG. 1, where modification of plastic supports PMMA (1) and/or PET (4) is carried out by treatment with an aqueous solution of monomethacrylamide derivative of 1,6-diaminohexane (2), and modified supports containing methacrylic functional groups are formed. The attachment of gel elements to these supports takes place by creation of covalent bonds during copolymerization of a gel forming mixture with participating methacrylic groups on the support. High efficiency of the modification procedure may be confirmed by affixing fluorescent labeled amines (6) onto methacrylated plastic supports. Fluorescent labeled amine (6) is used as a modifying agent for estimation of the level of modification instead of amine (2). After the modification procedure, the fluorescent signals are measured, such as by the method of Fixe et al. (2004).

Another embodiment of the methods described herein includes the step of washing or deep cleaning plastic supports. The plastic supports are washed by one or more organic solvents to improve the adhesive properties of the material. Selected solvents or combinations of solvents and treatment conditions for plastic slides improve the adhesive properties of the plastic surface, which provide attachment of gel elements to the support by adhesive forces.

Washing or deep cleaning procedures are carried out by the treatment of plastic surfaces illustratively for 2 hours at room temperature, or 1 hour at 60° C. with different organic solvents. Illustrative organic solvents include alcohols, such as ethanol, iso-propanol, and the like, alkanes, such as pentanes, hexanes, cyclohexane, methylcylclohexane, and the like, aromatic solvents, such as benzene, toluene, xylenes, and the like, and combinations thereof. In one aspect, benzene, toluene, and combinations thereof are used. The quality of gel drop microarrays produced by the methods described herein may be assessed visually and qualitatively by observing for example, the reproducibility of shape, size, and other physical features, and by tracking the failure resistance of the gel drop under assay conditions, and other visual assessments, either aided or unaided by magnification.

In another illustrative embodiment, an indicator such as an unsaturated carboxylic acid derivative of fluorescein, including FITC, is included in the gel drop microarray. Such an indicator may be used either qualitatively or quantitatively to evaluate the quality of and mechanical, storage, and other properties of the 3D gel drop array prepared as described herein.

It has been observed herein that such washing or deep cleaning of plastic surfaces with organic solvents significantly improves the adhesion properties of plastic material and allows surfaces to be used for biochip manufacturing without preliminary chemical modification of the surface. Without being bound by theory, a number of mechanisms for this adhesive behavior are suggested herein. In one aspect, the adhesive behavior may be due to the removal of dust, dirt, oils, unreacted monomer, or other material from the surface, thereby exposing a number of crevices, cracks, peaks, and valleys that will secure the polymerized gel-drop by a simple mechanical lock. In the case of crevices and valleys, the lock may be achieved by polymeric precursor filling such voids, then after polymerization, a mechanical lock is achieved. In the case of surface elements that protrude, lock may be achieved by polymeric precursor surrounding such protrusions, then after polymerization, a mechanical lock is achieved.

In another aspect, adhesion may be achieved by a swelling phenomenon of the plastic substrate once it is contacted with the solvents described herein. Polymeric materials may swell in the presence of certain solvents, and upon printing and subsequent polymerization of the gel forming mixtures, the surface swelling subsides and “grabs” the gel drop.

In another aspect, adhesion may be achieved by a phenomenon where the porosity of the plastic substrate is increased once it is contacted with the solvents described herein. Upon printing and subsequent polymerization of the gel forming mixtures, the porosity returns to normal levels and “grabs” the gel drop. It is appreciated that the porosity mechanism and the swelling mechanism may be related or even function in concert to achieve the adhesion of the printed and polymerized gel drop.

In another aspect, adhesion may be achieved by covalent attachment. Such covalent attachment may have been unachievable or inferior using conventional methods because washing or cleaning may be removing reactive monomer that forms the covalent bond but fails to attach itself or anything else to the surface, leading to poorer quality printing, and less stable biochips.

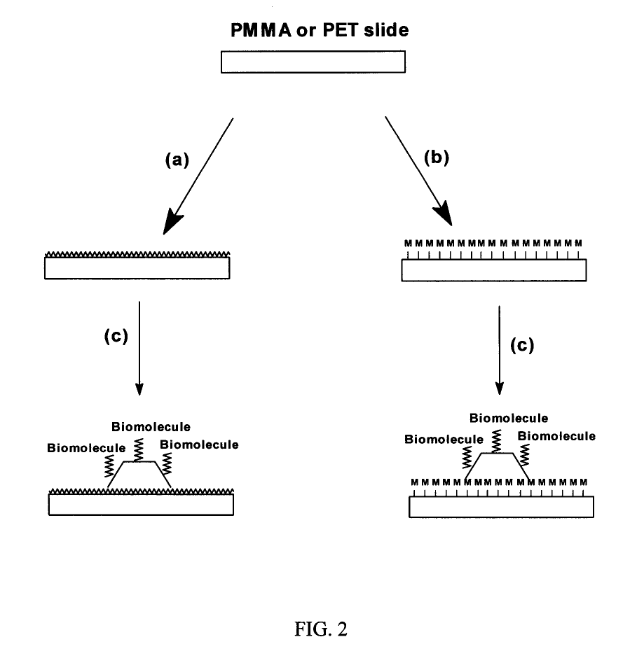

Additional exemplary embodiments are illustrated in FIG. 2 where biochip microarrays on plastic supports are produced by using gel-drop technology. In these embodiments, the plastic support may be prepared by washing or deep cleaning, or alternatively by chemical modification of the plastic support. The biomolecules are printed with conventional techniques and procedures using unpolymerized gel forming solutions, including optional cross-linking agents, mixed with biological probes, including but not limited to DNA fragments, proteins, peptides, and the like, as spots on the prepared plastic slides. After printing, the gel drops are polymerized, illustratively by UV exposure. FIG. 2 illustratively shows chemical modification with groups M in step (b). Alternatively, FIG. 2 shows surface modification by washing with an organic solvent in step (a).

In certain aspects described herein, the plastic supports may also exhibit improved mechanical properties, such as higher mechanical strength, higher impact resistance, higher toughness, and the like, then is exhibited by conventional glass supports. It is appreciated that such improved mechanical properties may be ideally suited for using the methods and biochips described herein for field use or clinical trials. It is also appreciated that the methods described herein may lead to more economically produced biochips. It is understood that many plastic supports cost less than comparable glass or silicon supports. In addition, there exist a large variation of plastic supports that are readily available from commercial suppliers. Accordingly, it is to be understood that the methods described herein may be modified with routine experimentation to be used with such a variety of plastic materials. Further, the biochips prepared by the methods described herein may be used in modules in more complicated and complex structures.

In one such complex structure, the biochips prepared by the methods described herein may be used to prepare or be incorporated into microfluidic devices. Such devices may require the improved mechanical properties described herein for plastic supports. Microfluidic devices represent a set of technologies that control the flow of micro, nano, or even picoliter amounts of liquids or gases in a miniaturized system.

In another embodiment, plastic microarray modules can be incorporated into different kinds of miniaturized microfluidic devices using biochip technology. Microfluidics refers to a set of technologies that control the flow of minute amounts of liquids or gases—typically measured in nano- and picoliters—in a miniaturized system. These technologies enable the construction of three-dimensional networks of channels and components, and they provide a high level of control over the molecular structure of channel surfaces. Over the past few years, microfluidic devices have enjoyed success in certain niche applications, notably lab-on-chip assays. Potential applications include pharmaceuticals, biotechnology, the life sciences, defense, public health, and agriculture, each of which has its own needs. Generally, microarrays prepared on the plastic surfaces described herein may include a larger variety of pathways or methods for surface modification, especially with different of chemical functional groups, than may be possible with conventional glass or silicon-based supports. Such multifunctional surfaces allow adhesive as well as covalent coupling of biomolecules.

In another embodiment, the plastic supports described herein may be used to prepare microfluidic devices. A microfluidic device can be identified by the fact that it has one or more channels with at least one dimension less than 1 mm. Common fluids used in microfluidic devices include whole blood samples, bacterial cell suspensions, protein or antibody solutions and various buffers. Potential applications include pharmaceuticals, biotechnology, the life sciences, national defense, public health, and agriculture, the needs of each of which being optimizable using routine methods. Microfluidic devices can also be used to obtain a variety of interesting measurements including molecular diffusion coefficients, fluid viscosity, pH, chemical binding coefficients and enzyme reaction kinetics, capillary electrophoresis, isoelectric focusing, immunoassays, flow cytometry, sample injection of proteins for analysis via mass spectrometry, PCR amplification, DNA analysis, cell manipulation, cell separation, cell patterning, chemical gradient formation, clinical diagnostics, and the like.

Materials and methods for fixing gel elements on PMMA and PET plastic surfaces may provide simple, cost-effective methods for their use as substrates in biochip manufacturing. Methacrylated plastic surfaces can be also used for making two dimensional biochips with bioprobes (oligonucleotides, peptides) modified with methacrylic function. After spotting of probes solutions on the plastic slides, covalent attachment of probes is carried out by applying of UV exposure providing radical polymerization reaction between methacrylic groups on the plastic surface and methacrylic functions on probe molecules.

In one aspect, the surface is modified with monomethacrylamide derivative of 1,6-diaminohexane. In another aspect, the surface is modified by treated by different organic solvents for washing or deep cleaning of the surfaces. In the first aspect modified surfaces are used for manufacturing 2D and 3D biochips. The second aspect is applicable just for 3D microarrays prepared by gel drops methods. The resulting biochips demonstrate good mechanical and thermal stability, as evidenced by their use as DNA-biochip for on-chip PCR experiments.

### EXAMPLES

1. Chemical modification of plastic supports with methacrylic groups. PMMA (Cat. No. ME303002,1; GoodFellow Corporation, PA, US) or PET (Cat. No. ES301450, GoodFellow Corporatopn. PA, US) supports embodied as slides (1×3 inches) were cleaned by washing with hexane (10 min) at room temperature, rinsing with MQ water and dried under vacuum. Plastic supports were incubated in 0.1 M solution of monomethacrylamide derivative of 1,6-diaminohexane in 0.1 M borate buffer (pH 11.2) during 2 h at room temperature. These modified supports were sequentially washed with 50% ethanol, MQ water, methanol and dried in a vacuum.

2. Washing plastic supports with organic solvents. PET (Cat.# ES301450, GoodFellow Corporatopn. PA, US) supports embodied as slides (1×3 inches) were incubated in toluene for 1 h at 60° C., then sequentially washed with 50% ethanol, MQ water, methanol and dried in a vacuum.

3. Detection of methacrylic groups on the plastic surface. For measuring of the density of methacrylic groups on the modified plastic slides, a reaction with FITC-labeled 1,6-diaminohexane (6) was performed. The fluorescence was detected by laser scanning of the surface (ScanArray Lite Microarray Analysis System; Packard bioscience, Billerica, Mass.). The density of methacrylic group was derived by comparing the fluorescent signal to a standard curve. The calibration curve was done by spotting on modified plastic slide by different concentration of fluorescent labeled hexamethylenediamine (6) and reading the fluorescence output signal for each dilution of the compound (6). A calibration curve was defined by plotting the fluorescence intensity as a function of the compound (6) concentration. The density of functional groups was found to be in the range of 0.2-0.3 nmol/cm². Additional details for this qualitative and quantitative determination of modification density are described in Fixe et al. (2004).

4. Fabrication of gel drop biochips. Copolymerization biochips were printed on plain plastic supports embodied as slides (1×3 inches). Copolymerization solutions that included the oligonucleotide probes were printed with a QArray2 arrayer (Genetix, New Milton, UK) using four “solid” 150 μm pins. A typical printing batch comprised 12 slides, each slide carrying four identical arrays of 400 drops. For the given slide-preparation protocol and the mixture composition, the diameter of drops was about 110 μm, which allowed printing arrays with a pitch of 300 μm both in rows and columns. On completion of printing, the slides were incubated during 1-1.5 h in an airtight container with 2 to 4 ml of a mixture that included all the components of the mixture used for printing the arrays except the oligonucleotides. After the incubation, the slides were placed in an airtight cassette equipped with quartz windows and polymerized for 30 min in a nitrogen atmosphere under a Thermo Spectronic Model XX-5A UV lamp (Cat. No. 11-982-120, Fisher Scientific, Pittsburgh, Pa.) that had its original 365 nm tubes changed for similar in design and electrical specifications 312 nm tubes Model FB-T1-110A (Fisher Scientific). Finally, the slides were transferred to an ARRAYIT High-Throughput Wash Station (Telechem International, Sunnyvale, Calif.) filled with 400 ml of 0.01M SSPE washing buffer (Ambion, Austin, Tex.), washed for 1 hour on a Nuova stirring hot plate (Barnstead/Thermolyne, Dubuque, Iowa), thoroughly rinsed with MilliQ water, and air dried. Additional details for preparing gel drops are found in using techniques similar to that described in Rubina A. et al. (2004).

DNA-biochips manufactured on plastic supports were tested for mechanical and thermal stability. Gel drops' biochips on plastic slides demonstrated failure resistance during washing procedures on an ARRAYIT High-Throughput Wash Station filled with of 0.01M SSPE washing buffer and after thoroughly rinsing with MilliQ water. Gel elements are strongly connected to the surface and survived during the washing procedure. They also can withstand 50-70 PCR cycles performed on MJ Research Peliter Thermal Cycler (applied conditions: 80° C., 2 min; 94° C., 5 min; then 50-70 cycles 94° C., 45 sec; 59° C., 90 sec; 72° C., 60 sec; 72° C., 5 min) without any damage (including changes in size, shape or loss of gel elements from the supports), or changing mechanical strength and optical transparency of the supports.

5. Testing of hybridization efficiency on gel drop arrays on plastic slides.

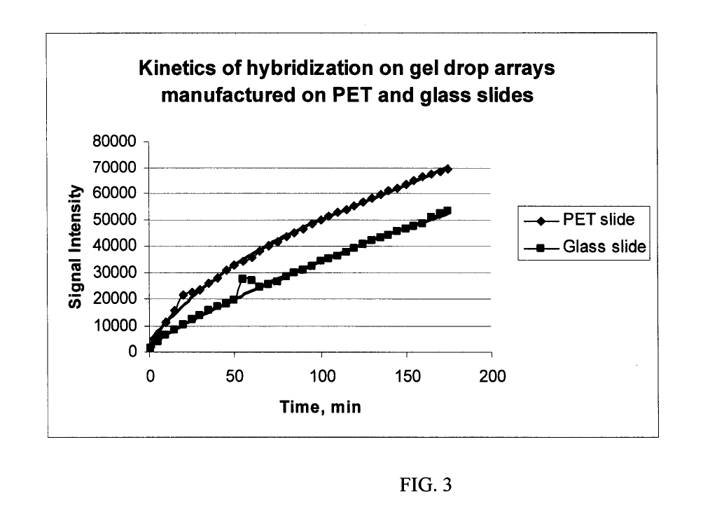

PET plastic slides after deep cleaning procedure have been examined in hybridization experiments on gel drop arrays. Copolymerization solutions were prepared containing 65% (w/w) glycerol, 4% (w/w) acrylamide-N,N-methylenebisacrylamide (19:1), 0.035 M sodium-phosphate buffer (pH 7.25) and 0.25 mM 3′methacrylated oligonucleotide. Copolymerization solutions were placed in 384-wells microtiter plates for microarray manufacturing. Gel drops arrays were manufactured as described herein. Hybridization was carried out in the buffer containing 0.01M sodium-phosphate (pH 7.2); 1 M sodium chloride; 1 mM EDTA and 0.1% (w/v) Tween 20; and 10 fmols/mkl of Texas Red-labeled oligonucleotide target. Hybridization was carried out in Frame-seal chambers (MJ Research) at room temperature. Measurements of fluorescent signals have been carried out on Argonne National Laboratory (ANL) stationary microscope, and signals obtained were processed with Microchip Imager software (ANL). Average fluorescent signals (background subtracted) were compared with the signals obtained from the same hybridization experiment carried out on gel drops array manufactured on glass slide. The results of the calculations are presented on FIG. 3.

### DOCUMENTS CITED

The following publications are also incorporated herein by reference to the extent that they relate to the conventional materials or methods described herein and useable with the invention described herein:
Arenkov, P., Kukhtin A., Gemmel A., Voloshchuk S., Chupeeva V. and Mirzabekov, (2000) A. Protein microchips: use for immunoassay and enzymatic reactions. Anal. Biochemistry, 278, 123-131.
Fixe et a. (2004). Functionalization of poly(methyl methacrylate) (PMMA) as a substrate for DNA microarrays, Nucleic Acids Res., 32(1,e9):1-8.
Han, J. et al. (2003). Ah.UV adhesive bonding technology in room temperature for plastic lab-on-a-chip. 7^{th} International Conference Miniaturized Chemical and Biochemical Analysis Systems, 10.5-9. CA, USA.
Ko, J. et al. (2003). A polymer-based microfluidic device for immunosensing biochips, Lab on a Chip, 3:106-113;
Mirzabekov A., Rubina A., Pan'kov S., Perov A., Chupeeva V., Composition for immobilization of biological macromolecules in hydrogels on forming biochips, method for preparation of the composition, biochip and method for carrying out PCR on a chip. PCT/RU 01/00445. priority 07.25.2001.
Mirzabekov A., Rubina A., Pan'kovs. And Chernov B. (2001). Method of immobilization of oligonucleotides, which containunsaturated groups, in polymeric hydrogels on the formation of microchip. Russian patent No. 2175972.
Reytavi R. et al. (2005) Microfluidic device for rapid (<15 min) automated microaraay hybridization. Clinical chemistry, 51:1836-1844.
Rubina A., Pan'kov S., Dementieva E., Pen'kov D., Butygin A., Vasiliskov V., Chudinov A., Mikhleikin A., Mikhailovich V, Mirzabekov A Hydrogel drop microchips with immobilized DNA: properties and methods for large-scale production. Anal. Biochemistry (2004) 325:92-106.
Rupcich N, et al. (2003) Coupled enzyme reaction microarray based on pin-printing of sol-del derived biomaterials, Analitica chimica Acta, 500:3-12.
Situma C. et al. (2005) Fabrication of DNA microarrays onto poly(methyl methacrylate) with ultraviolet patterning and microfluidics for the detection of low-abundant point mutations. Anal. Biochem., 430:123-135.
Starke V. et al (2006). Microarray technology for space exploration, LOCAD group report, NASA MSFC. Lunar and planetary Science XXXVII Meeting, USA.

1. A method for preparing a biochip on a plastic support, the method comprising:
(a) reacting the plastic support with an unsaturated carboxylic acid derivative having a primary amine functional group;
(b) printing one or more biomolecules on the plastic support; and
(c) covalently attaching the one or more biomolecules to the plastic support by polymerization.

2. The method of claim 1 wherein the unsaturated carboxylic acid derivative is an amide of acrylic or methacrylic acid.

3. The method of claim 1 wherein the primary amine functional group is covalently attached to the unsaturated carboxylic acid derivative through a bivalent linear or branched alkylene, cycloalkylene, alkenylene, alkynylene, or phenylene group, or a combination thereof.

4. The method of claim 1 wherein the primary amine functional group is covalently attached to the unsaturated carboxylic acid derivative through a bivalent alkylene group.

5. The method of claim 4 wherein the bivalent alkylene group is of the formula —(CH₂)ₙ—, where n is an integer from 2-10.

6. The method of claim 1 wherein the biochips are 2D biochips.

7. A method for preparing a biochip on a plastic support, the method comprising:
(a) reacting the plastic support with an unsaturated carboxylic acid derivative having a primary amine functional group;
(b) printing a microarray of gel forming mixtures loaded with one or more biomolecules on the plastic support; and
(c) covalently attaching the microarray to the plastic support by polymerization of the gel forming mixtures.

8. The method of claim 7 wherein the unsaturated carboxylic acid derivative is an amide of acrylic or methacrylic acid.

9. The method of claim 7 wherein the primary amine functional group is covalently attached to the unsaturated carboxylic acid derivative through a bivalent linear or branched alkylene, cycloalkylene, alkenylene, alkynylene, or phenylene group, or a combination thereof.

10. The method of claim 7 wherein the primary amine functional group is covalently attached to the unsaturated carboxylic acid derivative through a bivalent alkylene group.

11. The method of claim 10 wherein the bivalent alkylene group is of the formula —(CH₂)ₙ—, where n is an integer from 2-10.

12. A method for preparing a biochip on a plastic support, the method comprising:
(a) washing the plastic support with an organic solvent;
(b) printing a microarray of gel forming mixtures loaded with one or more biomolecules on the plastic support; and
(c) attaching the microarray to the plastic support by polymerization of the gel forming mixtures.

13. The method of claim 12 wherein the organic solvent is selected from the group consisting of ethanol, isopropanol, cyclohexane, benzene, toluene, and combinations thereof.

14. The method of claim 12 wherein step (b) includes one or more biomolecules having an unsaturated carboxylic acid functional group or amide derivative thereof.

15. The method of claim 14 wherein the unsaturated carboxylic acid functional group or amide derivative thereof is an acrylate or methacrylate derivative.

16. The method of claim 12 wherein the plastic support comprises poly(methylmethacrylate), poly(ethyleneterephthalate), or a combination thereof.

17. The method of claim 7 wherein the biochips are 3D biochips.

18. The method of claim 12 wherein the microarray is printed using a gel drop method.

19. The method of claim 12 wherein step (c) is performed in the presence of UV radiation.

20. A biochip comprising one or more biomolecules on a plastic support prepared by the method of claim 1.

21. A biochip comprising one or more biomolecules on a plastic support prepared by the method of claim 12.
